(19) United States
(12) Patent Application Publication
(10) Pub. No.: US 2026/0297508 A1
(43) Pub. Date: Oct. 1, 2026

(54) MODULATORS OF PROTEIN SECRETION

(71) Applicants: SANOFI, Paris (FR); INSTITUT NATIONAL DE LA SANTÉ ET DE LA RECHERCHE MÉDICALE, Paris (FR); SORBONNE UNIVERSITE, Paris (FR); UNIVERSITÉ PARIS CITÉ, Paris (FR)

(72) Inventors: Stephane BASMACIOGULLARI, Paris (FR); Mathilde COULET, Paris (FR); Guido KROEMER, Paris (FR); Oliver KEPP, Paris (FR)

(73) Assignees: SANOFI, Paris (FR); INSTITUT NATIONAL DE LA SANTÉ ET DE LA RECHERCHE MÉDICALE, Paris (FR); SORBONNE UNIVERSITE, Paris (FR); UNIVERSITÉ PARIS CITÉ, Paris (FR)

(21) Appl. No.: 19/483,041

(22) PCT Filed: May 10, 2024
(86) PCT No.: PCT/IB2024/054570

§ 371 Date: Nov. 10, 2025

(30) Foreign Application Priority Data
May 11, 2023 (EP) 23315197.6

(51) Int. Cl. C12N 5/00; C07K 16/28

(52) U.S. Cl. CPC C12N 5/0018; C07K 16/2827; C07K 2317/24; C12N 2501/999

(57) ABSTRACT

The present disclosure provides a method for identifying modulators of protein secretion. Also provided are modulators of protein secretion and use thereof.

### CROSS-REFERENCE TO RELATED APPLICATION

This application claims priority to EP Application Serial No. 23315197.6, filed May 11, 2023, the content of which is incorporated herein by its entirety.

### SEQUENCE LISTING

The instant application contains a Sequence Listing that has been submitted electronically in XML format and is hereby incorporated by reference in its entirety. Said XML copy, created on May 3, 2024, is named 122548WO034.xml and is 60,335 bytes in size.

### BACKGROUND

Protein secretion is a biological process fundamental to cell growth, movement, and communication. Many regulatory mechanisms act as quality control for this process. Defects in protein folding and secretion can lead to various pathologies such as cancer and neurodegenerative, metabolic and inflammatory diseases.

Understanding of the cellular secretion mechanisms is also of paramount importance to the production of biologic therapeutics. The number of protein therapeutics made from cultured cells has greatly increased in the past two decades, representing today one quarter of all approved drugs, with about half of them being antibody therapeutics (Walsh and Walsh, Nat Biotechnol. (2022) 40:1722-60). Many approaches have been attempted to optimize titers of recombinant proteins, including host cell selection, vector design, and culture medium formulation.

Conversely, uncontrolled protein secretion might induce cell dysfunction and pathogenesis. See, e.g., Kuo et al., Cell Sys. (2021) 12 (9): 873-84) and Wang et al., Mol Neurodegen. (2014) 9:31). In cases where secreted molecules have deleterious effects on neighboring cells, uncontrolled secretion may lead to cell death, which is of concern in bioproduction.

Thus, there remains a need to identify approaches to modulate protein secretion and to optimize production of biologics.

### SUMMARY

The present disclosure provides modulators of protein secretion from eukaryotic cells. Accordingly, in one aspect, the present disclosure provides a method of inhibiting protein secretion from a eukaryotic cell, comprising contacting the cell with nigericin or tyrphostin AG-879, or an analog thereof. In some embodiments, the cell is a in a cell culture (i.e., in vitro). In some embodiments, the contacting comprises adding nigericin, tyrphostin AG-879, or analog to the cell culture. In further embodiments, the nigericin, tyrphostin AG0879, or analog is added to the cell culture to reach a concentration of about 1 μg/mL to about 100 μg/mL, optionally about 5 μg/mL to about 50 g/mL. In further embodiments, the nigericin, tyrphostin AG0879, or analog is added to the cell culture to reach a concentration of about 50 μg/mL. In further embodiments, the nigericin, tyrphostin AG0879, or analog is added to the cell culture to reach a concentration of at least about 50 μg/mL.

In another aspect, the present disclosure provides a method of increasing protein secretion from a eukaryotic cell, comprising contacting the cell with lycorine or an analog thereof. In some embodiments, the eukaryotic cell is a recombinant cell engineered to express a recombinant protein, such as an enzyme for enzyme replacement therapy, or an antibody. In some embodiments, the cell is in a cell culture. In some embodiments, the contacting comprises adding lycorine or analog to the cell culture. In further embodiments, the lycorine or analog is added to the cell culture to reach a concentration of from about 0.1 μM to about 100 μM, optionally from about 1 μM to about 50 μM. In further embodiments, the lycorine or analog is added to the cell culture to reach a concentration of at least about 1 μM. In further embodiments, the lycorine or analog is added to the cell culture to reach a concentration of about 1 μM. In further embodiments, the lycorine or analog is added to the cell culture to reach a concentration of about 50 μM.

In some embodiments of the present methods, the eukaryotic cell is a mammalian cell, an insect cell, or a yeast cell. In further embodiments, the mammalian cell is a CHO cell, a NSO cell, a BHK cell, a 293 cell, a HeLa cell, a HEK cell, or a Sp2/0 cell.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1A is a schematic diagram showing the principle of the RUSH assay for antibody secretion. The fusion protein (in this case, the antibody fused to the streptavidin-binding domain (SBD) separated by a linker that contains a furin cleavage site) is retained in the endoplasmic reticulum (ER) lumen via the interaction of SBD with a streptavidin-KDEL hook protein. Upon addition of biotin, the interaction between the hook and the fusion protein is competitively disrupted and the fusion protein is hence free to journey through the classical secretory pathway to the Golgi, where the resident furin protease cleaves the linker between SBD and the antibody. The resulting parts are then secreted into the extracellular environment. The GFP moiety enables the monitoring of protein trafficking by fluorescence microscopy.

FIG. 1B is a schematic diagram showing an exemplary RUSH antibody. The antibody is composed of an IgG fused at its heavy chain C-termini to a peptide sequence containing a furin-cleavable linker, a green fluorescent protein (GFP) and a streptavidin-binding peptide (SBP).

FIG. 1C is a panel of photographs of immuno-stained fixed cells showing the colocalization of the streptavidin-KDEL hook with calreticulin in the ER. Pearson's correlation coefficient (PCC)=0.69. Surface overlap coefficient (SOC)=0.45.

FIGS. 2A-2F show characterization of the anti-PD-L1 antibody RUSH system. FIG. 2A shows time lapse microscopy of clone #7 cells incubated in absence or in presence of 40 μM biotin, fixed and stained with DAPI. The secretion rate can be deducted from the decreasing intracellular GFP intensity upon addition of biotin to the cell cultures (FIG. 2A). FIG. 2B shows immunostaining of fixed cells incubated for 4 h in the absence or in the presence of biotin showing the colocalization of the streptavidin-KDEL hook with the GFP-tagged antibody in the absence of biotin and the loss of colocalization 4 h post biotin addition. PCC=0.51 in the absence of biotin, PCC=0.29 in the presence of biotin. Western blot of cell lysates (FIG. 2C) or concentrated supernatants (FIGS. 2D-2F) of clone #7 incubated for 4 h in the absence of biotin, in the presence of 40 μM biotin, or in the presence of 40 μM biotin together with 100 μM furin inhibitor I. Membranes were probed with the indicated antibodies to detect proteins that contain the GFP moiety (FIGS. 2C, 2D), the SBP moiety (FIG. 2E) or epitopes from the immunoglobulin heavy and light chains (FIG. 2F). Molecular mass standards (in kDa) are indicated on the left in panel FIG. 2D. The structures of the proteins detected by immunoblot are indicated based on the scheme shown in FIG. 1B and include antibody heavy and light chains, GFP, SBP, and a linker with a furin cleavage site. The truncated shape at the antibody C-terminus represents linker residues left after cleavage by furin. Results are representative of at least 3 independent experiments.

FIGS. 3A-F show the characterization of the binding capacity of the anti-PD-L1 RUSH antibody released through the biotin-activated RUSH system (FIGS. 3A, 3C, and FIG. 3E) Schematic representation of the antibody binding to PD-L1^{pos} cells and detection by flow cytometry. FIG. 3B shows a FACS histogram plot of native recombinant and purified anti-PD-L1 antibody. FACS histogram plots of processed and unprocessed RUSH anti-PD-L1 antibody released by clone #7 in the presence of biotin and in the absence or presence of furin inhibitor, respectively (FIGS. 3D, 3F). Dark grey, binding to PD-L1^{pos} CHO cells (CHO-PDL1); light grey, binding to PD-L1^{neg} parental CHO cells (CHO-K1). Antibody binding is detected by means of an indirect immunofluorescence assay with a secondary anti-Fc APC conjugated or by direct measurement of GFP fluorescence.

FIGS. 4A-4C show the high-throughput screening of chemical compound libraries for the identification of modulators of protein secretion. FIG. 4A shows screening workflow. U2OS clone #7 is plated in 384-well plates and incubated for 24 h before treatment with each compound of the Prestwick, ICCB Known Bioactives, or Autophagy Compound Library at 2 concentrations, low and high (with a 10-fold difference). Biotin is added after 4 h, cells are washed and fixed 1 h after addition of biotin, and processed for automated image acquisition of the intracellular Hoechst 33324 and GFP fluorescence. FIG. 4B shows a dot plot representation of drugs based on their impact on GFP release (cytoplasmic GFP signal) and nucleus integrity (viability). FIG. 4C shows Z-score ranking of the cytoplasmic GFP intensity after treatment. The results of the Prestwick library screening at the low drug concentration are plotted. Brefeldin A was used as a positive control of the inhibition of secretion. The compounds causing a cytoplasmic GFP intensity below that observed for biotin-only treated cells are potential enhancers of protein secretion. Drugs that induce a GFP fluorescence level higher than that measured in the presence of biotin only are potential inhibitors of protein secretion.

FIGS. 5A-5C show selection of potential enhancers of protein secretion. FIGS. 5A and 5B) show dot plot representations of drugs efficiency based on their Z-scores measured at low and high drug concentration (FIG. 5A, Prestwick library; FIG. 5B, ICCB Known Bioactives and TargetMol Autophagy libraries). Compounds selected for further characterization are listed. (FIG. 5C) Clone #7 was treated with the indicated compounds, fixed and analyzed as described in FIG. 4A legend.

FIG. 6A-6C show validation of enhancers of protein secretion. Clone #7 cells were treated with the indicated compounds and analyzed as indicated in FIG. 4A legend. Drugs were used at low (FIG. 6A) or high concentration (FIG. 6B) as indicated in the text and post treatment intracytoplasmic fluorescence was normalized to that measured in mock treated cells (no biotin). FIG. 6C shows an ELISA assay on immobilized PD-L1.

FIGS. 7A and 7B show validation of inhibitors of protein secretion. FIG. 7A shows ELISA assay on immobilized PD-L1. Clone #7 cells were treated with the indicated compounds and supernatants were assayed for PD-L1 binding. Brefeldin A was used as a protein secretion inhibitor positive control. FIG. 7B shows cells treated in FIG. 7A were subjected to a toxicity assay. Hoechst 33324 fluorescence was measured, which enabled to number adherent living cells in each well.

### DETAILED DESCRIPTION

The present disclosure provides a method for identifying modulators of antibody secretion from recombinant host cells by leveraging the Retention Using Selective Hooks (RUSH) system in medium- to high-throughput drug library screening.

The RUSH system is based on the retention of the protein of interest in a specific subcellular location until its triggered-release in the secretory pathway. The system utilizes a two-state system where a hook protein fused to core streptavidin is firmly anchored in the donor compartment, and it interacts reversibly with a reporter protein fused to a streptavidin-binding peptide (SBP). Upon the addition of biotin, the reporter protein is simultaneously released from the hook protein. See, e.g., Boncompain et al., Nat Methods. (2012) 9:493-98; Zhao et al., Sci Rep. (2018) 8:14966; WO 2022/101482.

Accordingly, the present disclosure provides a method for identifying a compound that modulates antibody secretion from a eukaryotic cell, the method comprising: providing a cell that has been engineered to express (i) a hook protein that is retained in the endoplasmic reticulum of the cell, wherein the hook protein comprises a biotin-binding domain (e.g., streptavidin), and (ii) a bait protein comprising an antibody whose heavy chain C-terminus is fused to a hook protein-binding peptide (e.g., a streptavidin-binding peptide); contacting the cell with a candidate compound (e.g., a member of a small molecule library) and with biotin, and determine the amount of said antibody secreted from the cell, wherein an altered amount of the secreted antibody from the cell as compared to a reference cell not contacted with the candidate compound indicates that the candidate compound is a compound capable of modulating antibody secretion from the cell.

The present inventors have generated an antibody-based RUSH system that allows for screening of modulators of protein (e.g., an antibody, an enzyme, or another protein biologic) secretion from cells that have been engineered to produce recombinant protein (e.g., an antibody, an enzyme, or another protein biologic). Screening of small compound libraries led to the discovery of lycorine, anisomycin, cycloheximide, digoxigenin, methenamine, ropinirole, pinocembrin, and pyrithyldione as secretion enhancers; and nigericin and tyrphostin AG-879 as secretion inhibitors. These compounds can be used to directly modulate protein secretion through their addition in the cell culture medium. For example, enhancers of protein secretion may be used to boost the yield of recombinant protein production, whereas inhibitors of protein secretion may be used to control the timing of protein secretion or to prevent or reduce secretion of proteins that are harmful.

### I. RUSH System

The present disclosure provides a cell-based assay (RUSH system) for identifying regulators of antibody secretion. In particular embodiments, the cell-based assay makes use of a cell line that co-expresses a hook protein and a bait protein to aide in the identification of small molecule modulators of protein secretion, and to monitor kinetics of protein secretion.

The cell line selected for the RUSH assay may be any eukaryotic cell line. The RUSH cell line may be the same as the cell line that is intended for the production of a target antibody. In some embodiments, the eukaryotic cell is a yeast cell. In some embodiments, the yeast cell is S. cerevisiae, P. pastoris, and the like. In some embodiments, the eukaryotic cell is an insect cell. In some embodiments, the eukaryotic cell is a mammalian cell. In further embodiments, the mammalian cell is a CHO cell, a HEK cell, a BHK cell, a 293 cell, a HeLa cell, a NSO cell, a Sp2/0 cell, a U2OS cell, and the like.

### A. Hook Protein

In some embodiments, the hook protein provided herein comprises a biotin-binding protein fused to a peptide that can sequester the hook protein in the endoplasmic reticulum (ER). Exemplarv ER-retention peptides are shown in the table below.

TABLE 1
Exemplary ER-Retention Peptides

| SEQ ID NO | Amino Acid Sequence |
|---|---|
| 3 | KDEL |
| 4 | KTEL |
| 5 | HNEL |
| 6 | KEDL |
| 7 | HDEL |
| 8 | KEEL |
| 9 | QEDL |
| 10 | K(X)KXX, wherein X is any amino acid residue and |
| | parenthesis |
| | indicate presence or absence. |
| 11 | HKXK, wherein X is any amino acid residue |
| — | RR, wherein X is any amino acid residue |
| — | RXR, wherein X is any amino acid residue |
| 12 | RXXR, wherein X is any amino acid residue |
| 13 | EFFDAXE, wherein X is any amino acid residue |
| — | DXE, wherein X is any amino acid residue |
| — | DIE |
| — | SKK |
| 14 | MAGARRGWRLAPVRRGVCGPRARPLMRPLWLLFAVSFFGWTGALDG |
| 15 | MGSLWSKISQLFVDA |
| 16 | MRVKEKYQHLWRWGWRWGTMLLGMLMIC |
| 17 | MRLSWFRVLTVLSICLSAVAT |
| 18 | MDPVVVLGLCLSCLLLLSLWKQSYGGGKL |
| 19 | HALSYWKPFLVNMCVATVLTAGAYLCYRFLFNSNT |
| 20 | SPRGSRPSWGPTDPRRRSRNLGKVIDTLTCGFADLMGYIPLVGA |
| 21 | MALWMRFLPLLALLVLWEPKPAQA |
| 22 | MEAMWLLCVALAVLAWG |
| 23 | IPHDLCHNGEKSKKPSKIKSLFKKKSK |
| 24 | RPSNNNTTTTTTTDATQPHHHHHHHRHRDAGVKNVTNNSKTTESSSSSSA |
| | AKEKPKHKKGLLHKLKKKL |
| 25 | VVQAITFIFKSLGLKCVQFLPQVMPTFLNVIRVCDGAIRE |
| 26 | TFCSTALLITALALVCTLLYL |
| 27 | GVMLGSIFCALITMLGHI |
| 28 | FLAAFVVMLCITIVTIVVYIYLV |
| 29 | WGVLAGIAYFSMVGNWAKVLVVLLLFAGV |
| 30 | TAPASINTPN |
| 31 | WWQLTLGAICALPLAGLLACCA |

In some embodiments, the ER-retention peptide comprises a KDEL (SEQ ID NO: 3), K(X)KXX (SEQ ID NO:10), RR, RXR, or RXXR (SEQ ID NO:12) motif, wherein X is any amino acid residue. In further embodiments, the ER-retention peptide comprises or consists of KDEL (SEQ ID NO:3).

In some embodiments, the biotin-binding protein is selected from avidin, streptavidin, tamavidin, bradavidin, extravidin, rhizavidin, and derivatives thereof. In further embodiments, the biotin-binding protein is streptavidin. An exemplary sequence of streptavidin is shown below:

(SEQ ID NO: 2)
MRKIVVAAIA VSLTTVSITA SASADPSKDS KAQVSAAEAG
ITGTWYNQLG STFIVTAGAD GALTGTYESA VGNAESRYVL
TGRYDSAPAT DGSGTALGWT VAWKNNYRNA HSATTWSGQY
VGGAEARINT QWLLTSGTTE ANAWKSTLVG HDTFTKVKPS
AASIDAAKKA GVNNGNPLDA VQQ

A variant protein that is at least 90% (e.g., at least 91, 92, 93, 94, 95, 96, 97, 98, or 99%) identical to this exemplary sequence may also be used, as long as the variant protein retains the ability to bind biotin or a derivative thereof.

The percent identity of two amino acid sequences (or of two nucleic acid sequences) may be obtained by, e.g., BLAST® using default parameters (available at the U.S. National Library of Medicine's National Center for Biotechnology Information website). In some embodiments, the length of a reference sequence aligned for comparison purposes is at least 30%, (e.g., at least 40, 50, 60, 70, 80, or 90% of the reference sequence.

In certain embodiments, the hook protein comprises streptavidin fused to a KDEL (SEQ ID NO:3) peptide.

The hook protein may be introduced to a host cell by transfecting an expression cassette encoding the hook protein into the host cell. The expression cassette may comprise a promoter that is active in the host cell. For mammalian host cells, for example, a promoter selected from CMV, SFFV, CAG, EFI, EFIA, GALI, GALI0, GPD, ADH and GAP promoters may be used.

### B. Bait Protein

The bait protein used in the present methods comprises an antibody, or another multimeric protein, fused to a peptide that can bind the hook protein. In some embodiments, the hook protein-binding peptide is a streptavidin-binding peptide (SBP) if the hook protein comprises streptavidin. Examples of SBP are shown in the table below. In some embodiments, the SBP comprises or consists of SEQ ID NO:32.

TABLE 2
Exemplary SBP Sequences

| SEQ ID NO | Amino add sequence |
|---|---|
| 32 | DEKTTGWRGGHVVEGLAGELEQLRARLEHHPQGQREP |
| 33 | AWRHPQFGG |
| 34 | AWRHPQFGGAWRHPQFGG |
| 35 | AWRHPQFGGAWRHPQFGGAWRHPQFGG |
| 36 | WSHPQFEK |
| 37 | WSHPQFEKWSHPQFEK |
| 38 | WSHPQFEKWSHPQFEKWSHPQFEK |
| 39 | WSGPQFEK |
| 40 | WSGPQFEKWSGPQFEK |
| 41 | WSGPQFEKWSGPQFEKWSGPQFEK |
| 42 | WHRWPWLVSG |
| 43 | WHRWPWLVSGWHRWPWLVSG |
| 44 | WHRWPWLVSGWHRWPWLVSGWHRWPWLVSG |
| 45 | WHWWYWALDR |
| 46 | WHWWYWALDRWHWWYWALDR |
| 47 | WHWWYWALDRWHWWYWALDRWHWWYWALDR |
| 48 | WX₁HPQFX₂X₃, wherein X₁ represents any amino |
| | acid residue, and X₂ and X₃ both represent G, |
| | or where X₂ represents E and X₃ |
| | represents R or K |
| — | DVE |
| — | VEA |
| — | EAW |
| 49 | VEAW |
| 50 | DVEA |
| 51 | DVEAW |
| 52 | DVXAW, wherein X represents E, D or G |
| 53 | DVXAWL, wherein X represents E, D or G |
| 54 | DVEAWL |
| 55 | DVX₁AWLX₂, wherein X₁ represents E, D or G; and |
| | X₂ represents G or D |
| 56 | DVX₁AWLX₂X₃, wherein X₁ represents E, D or G; X₂ |
| | represents G or D; and X₃ represents A or E |
| 57 | DVX₁AWLX₂X₃R, wherein X₁ represents E, D or G; |
| | X₂ represents G or D; and X₃ represents A or E |
| 58 | DVEAWLGAR |
| 59 | DVX₁AWLX₂X₃RVPLVET, wherein X₁ represents E, D |
| | or G; X₂ represents G or D; and X₃ represents A |
| | or E |
| 60 | DRATPY |
| 61 | SEKLSNTK |

The multimeric protein may be an antibody (e.g., an IgG1, IgG2, IgG3, or IgG4) whose heavy chain C-termini are fused to an SBP. The antibody may bind to any antigen of interest, including, without limitations, cytokines (e.g., TNF-alpha, IL-6, or TGF-beta), tumor antigens (e.g., AFP, BCMA, CD19, CD20, CD22, CD123, EGFR, EpCAM, GPC2, GPC3, HER2, MUC16, ROR1, or ROR2), or immune checkpoints (e.g., PD-1, PD-L1, PD-L2, CTLA-4, TIGIT, TIM-3, or LAG-3).

In some embodiments, the bait protein may further comprise a fluorescent signal domain (e.g., a green fluorescent protein (GFP) domain or enhanced GFP (eGFP)) domain to allow the monitoring of the trafficking of the bait protein within the cell. In such embodiments, the fluorescent signal domain may be linked to the multimeric protein (e.g., the antibody) through a linker cleavable by a protease in a subcellular compartment, such as a furin resident in the Golgi apparatus. A furin cleavage site may be RX₁X₂R, where X₁=any naturally occurring amino acid, and X₂=R or K (SEQ ID NO:62).

The bait protein may be introduced to a host cell by transfecting an expression cassette encoding the bait protein into the host cell. The expression cassette may comprise a promoter that is active in the host cell. For mammalian host cells, for example, a promoter selected from vav, PGK, SV40, thymidine kinase promoter (TK), MSCV, and UbC promoters may be used.

### C. Peptide Linkers

In the hooking protein and the bait protein, the various functional domains may be linked through a peptide linker. The peptide linkers may be flexible linkers so as to allow for proper folding, movement, and interaction of the joined domains. In some embodiments, the flexible peptide linker herein largely comprises small amino acids (e.g., Gly, Ser, or Thr). In some embodiments, the peptide linker herein consists primarily (e.g., more than 50% of the residues) of Gly and Ser residues (“GS” linker). As described above, such a peptide linker may comprise (G₄S)ₙ (SEQ ID NO:1). By adjusting the copy number “n,” the length of the linker can be adjusted to achieve the desired distance of the joined functional domains. In some embodiments, the n may be 1, 2, 3, 4, 5, 6, 7, 8, 9, or 10. In further embodiments, the n may be 2 or 3. In some embodiments, the peptide linker may contain additional amino acids such as Thr and Ala to maintain flexibility, as well as polar amino acids such as Lys and Glu to improve solubility. See, e.g., Chen et al., Adv Drug Deliv Rev. (2013) 65(10):1357-69.

In some embodiments of the hook protein, the streptavidin domain and the ER-retention domain (e.g., the KDEL peptide) may be linked a GS linker such as (G₄S)ₙ (SEQ ID NO: 1), where n may be, for example, 2 or 3 (i.e. GGGGGGGGS or GGGGSGGGGSGGGGS, respectively).

In some embodiments of the bait protein, an IgG is fused at its C-termini to an SBP through a peptide linker. In further embodiments, the peptide linker is cleavable. In certain embodiments, the peptide linker includes a furin cleavage site and is cleavable by furin. In certain embodiments, the cleavable (e.g., furin-cleavable) linker links the IgG to a fluorescent signal peptide and an SBP.

### D. Biotin and Derivatives Thereof

Biotin is added to the RUSH cells to disrupt the binding between the hook protein and the bait (antibody) protein. In some embodiments, a biotin derivative may be used. In some embodiments, a biotin derivative has the structure of Formula (I):

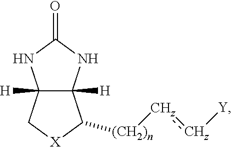

wherein:
X is selected from H₂, O, S, Se, SO, and SO₂,
Y is selected from CONH(CH₂)₄CH(NH₂) COOH, COOH, and OH,
n is 1, 2 or 3, and
z is 1 or 2.

In one embodiment, the biotin derivative is selected from the group consisting of biocytin, dethiobiotin, selenobiotin, biotin sulfoxide, oxybiotin, biotinol, norbiotin, homobiotin, alpha-dehydrobiotin, and biotin sulfone.

### II. Identification of Modulators of Protein Secretion

The present disclosure provides methods of screening for modulators (agonists or antagonists) of protein (e.g., multimeric protein such as an antibody) secretion. In these methods, candidate compounds (e.g., those in a small molecule library) are added to cells comprising the antibody RUSH system described herein. With the addition to biotin (or a derivative thereof) to the cells, the antibody bait will be released from sequester in the ER. Measurement of antibody secretion can be performed (e.g., by ELISA) to determine alteration of antibody secretion relative to reference cells that are not treated with the candidate compounds. The candidate compounds may be added prior to, concurrent with, or after, the addition of biotin to the cell culture.

The screening methods of the present disclosure allow a compound (e.g., a small molecule compound) to be identified as being an enhancer or inhibitor of protein secretion, e.g., antibody secretion.

### III. Small Molecule Modulators of Antibody Secretion

The present disclosure also provides enhancers and inhibitors of protein (e.g., monomeric protein or multimeric protein such as antibody) secretion identified by the present screening methods.

### A. Enhancers of Protein Secretion

In some embodiments, the enhancer of protein (e.g., antibody) secretion is lycorine (3,12-Didehydro-2′H-[1,3]dioxolo[4′,5′:9,10]galanthan-1α,2β-diol). Lycorine is a natural alkaloid extracted from the Amaryllidaceae plant. Lycorine is a potent and orally active SCAP inhibitor. Lycorine downregulates the SCAP (SREBP cleavage activating protein; SREBP, sterol regulatory element-binding protein) protein level without changing its transcription. Lycorine exhibits potential as a melanoma vasculogenic inhibitor, making it useful in the study of metabolic diseases and prostate cancer. See, e.g., Roy et al., Biomed Pharmacother. (2018) 107:615-24. Lycorine is identified by CAS No. 476-28-8 and has the following chemical structure:

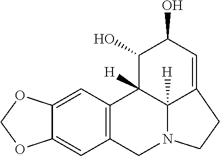

In some embodiments, the enhancer of protein (e.g., antibody) secretion is anisomycin ([(2R,3S,4S)-4-hydroxy-2-[(4-methoxyphenyl)methyl]pyrrolidin-3-yl]acetate). Anisomycin is a potent inhibitor of protein synthesis and interferes with protein and DNA synthesis by inhibiting peptidyl transferase or the 80S ribosome system. Anisomycin acts as a JNK activator, boosting phospho-JNK levels. Anisomycin is also a bacterial antibiotic. Anisomycin is identified by CAS No. 22862-76-6 and has the following chemical structure:

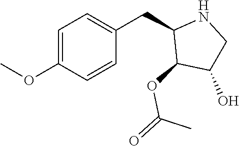 [SMILES: COc1ccc(C[C@H]2NC[C@H](O)[C@H]2OC(C)=O)cc1]

In some embodiments, the enhancer of protein (e.g., antibody) secretion is cycloheximide (4-[(2R)-2-[(1S,3S,5S)-3,5-dimethyl-2-oxocyclohexyl]-2-hydroxyethyl]piperidine-2,6-dione). Cycloheximide is a dicarboximide that is 4-(2-hydroxyethyl) piperidine-2,6-dione in which one of the hydrogens attached to the carbon bearing the hydroxy group is replaced by a 3,5-dimethyl-2-oxocyclohexyl group. It is an antibiotic produced by the bacterium Streptomyces griseus, and functions as a bacterial metabolite, a protein synthesis inhibitor, a neuroprotective agent, an anti-coronaviral agent, and a ferroptosis inhibitor. It is a member of piperidones, a piperidine antibiotic, an antibiotic fungicide, a dicarboximide, a secondary alcohol and a cyclic ketone, and it is functionally related to a piperidine-2,6-dione. Cycloheximide is identified by CAS No. 17974-04-8 and has the following chemical structure:

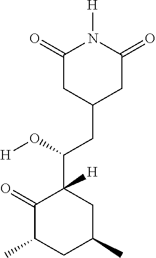 [SMILES: C[C@@H]1C[C@@H]([C@H](O)CC2CC(=O)NC(=O)C2)C(=O)[C@@H](C)C1]

In some embodiments, the enhancer of protein (e.g., antibody) secretion is digoxigenin (3-[(3S,5R,8R,9S,10S,12R,13S,14S,17R)-3,12,14-trihydroxy-10,13-dimethyl-1,2,3,4,5,6,7,8,9,11,12,15,16,17-tetradecahydrocyclopenta[a]phenanthren-17-yl]-2H-furan-5-one). Digoxigenin is a hydroxy steroid that consists of 5-beta-cardanolide having a double bond at the 20(22)-position as well as hydroxy groups at the 3-beta-, 12-beta- and 14-beta-positions. It has been isolated from the plant species of the genus Digitalis, and functions as a hapten and a plant metabolite. Digoxigenin is identified by CAS No. 1672-46-4 and has the following chemical structure:

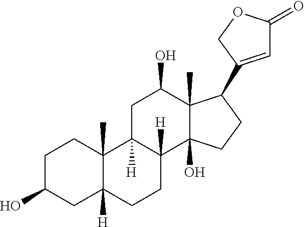

In some embodiments, the enhancer of protein (e.g., antibody) secretion is methenamine (1,3,5,7-tetrazatricyclo[3.3.1.13,7]decane). Methenamine is a heterocyclic organic compound with antibiotic activity. In the body, methenamine is converted to formaldehyde, which is a nonspecific bactericidal agent. Methenamine is typically used long-term to treat chronic urinary tract infections and to prevent the recurrence of infections. Methenamine is identified by CAS No. 37604-90-3 and has the following chemical structure:

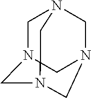

In some embodiments, the enhancer of protein (e.g., antibody) secretion is ropinirole (4-[2-(dipropylamino)ethyl]-1,3-dihydroindol-2-one). Ropinirole (SKF 101468) is an orally active, potent D₃/D₂ receptor agonist, but with no affinity for the D1 receptors. Ropinirole is identified by CAS No. 91374-21-9 and has the following chemical structure:

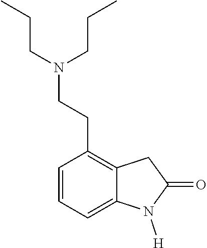 [SMILES: CCCN(CCC)CCc1cccc2c1CC(=O)N2]

In some embodiments, the enhancer of protein (e.g., antibody) secretion is pinocembrin (5,7-Dihydroxy-2-phenyl-2,3-dihydro-4H-chromen-4-one). Pinocembrin is a compound found in Pinus contorta var. latifolia, and Piper nigrum. Pinocembrin is identified by CAS No. 68745-38-0 and has the following chemical structure:

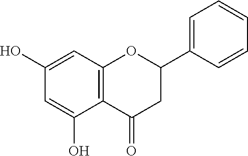 [SMILES: O=C1CC(c2ccccc2)Oc2cc(O)cc(O)c21]

In some embodiments, the enhancer of protein (e.g., antibody) secretion is pyrithyldione (3,3-diethyl-1H-pyridine-2,4-dione). Pyrithyldione is a psychoactive drug, and is identified by CAS No. 77-04-3 and has the following chemical structure:

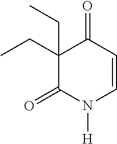

Analogs of the above-exemplified enhancers may also be used. By “analog” is meant a compound that is a functional equivalent to a reference compound and shares the same or similar core chemical structure. An analog of a compound includes, for example, a pharmaceutically acceptable salt or ester of the compound.

In some embodiments, an enhancer herein (e.g., lycorine or an analog thereof) is added to the cell culture medium to reach a concentration of about 0.1 μM to about 1000 μM (e.g., about 0.5 μM to about 100 μM, or about 1 μM to about 50 μM). In some embodiments, lycorine (or an analog thereof) is added to the cell culture medium to reach a concentration of about 1 μM or about 50 μM. In some embodiments, lycorine (or an analog thereof) is added to the cell culture medium to reach a concentration of about 1 μM. In some embodiments, lycorine (or an analog thereof) is added to the cell culture medium to reach a concentration of about 50 μM. In some embodiments, lycorine (or an analog thereof) is added to the cell culture medium to reach a concentration of at least about 1 μM.

### B. Inhibitors of Protein Secretion

In some embodiments, the inhibitor of protein (e.g., antibody) secretion is nigericin. Nigericin is an antibiotic derived from Streptomyces hygroscopicus that functions as a K+/H+ ionophore, promoting K+/H+ exchange across mitochondrial membranes. Nigericin can also act as a NLRP3 (nucleotide-binding domain leucin-rich repeat [LRR] and pyrin-containing receptor 3) activator, leading to the release of IL-1β. Nigericin has been found to promote eryptosis, a process that is accompanied by the formation of ROS and is partly attributed to oxidative stress induction. Additionally, nigericin triggers apoptosis. Nigericin is identified by CAS No. 28380-24-7 and has the following chemical structure:

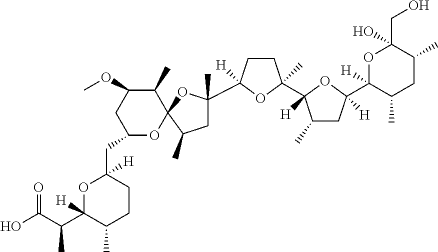 [SMILES: CO[C@@H]1C[C@@H](C[C@H]2CC[C@H](C)[C@H]([C@@H](C)C(=O)O)O2)O[C@]2(O[C@](C)([C@H]3CC[C@@](C)([C@@H]4O[C@@H]([C@H]5O[C@@](O)(CO)[C@H](C)C[C@@H]5C)C[C@@H]4C)O3)C[C@H]2C)[C@@H]1C]

In some embodiments, the inhibitor of protein (e.g., antibody) secretion is tyrphostin AG 879 or “AG 879.” AG 879 is a tyrosine kinase inhibitor that effectively inhibits TrKA phosphorylation and has no inhibitory effect on TrKB and TrKC. This compound is also a selective inhibitor of ErbB2 tyrosine kinase, with at least 500-fold greater specificity to ErbB2 than to EGFR. AG 879 has also demonstrated anticancer activity. AG 879 is identified by CAS No. 148741-30-4 and has the following chemical structure:

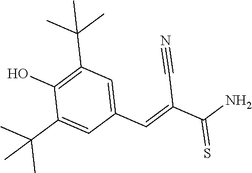 [SMILES: CC(C)(C)c1cc(/C=C(\C#N)C(N)=S)cc(C(C)(C)C)c1O]

Analogs of the above-exemplified inhibitors may also be used. In some embodiments, an inhibitor herein (e.g., nigericin or AG 879, or an analog thereof) is added to the cell culture medium to reach a concentration of about 0.5 μg/mL to about 500 μg/mL (e.g., about 1 μg/mL to about 100 μg/mL, about 5 μg/mL to about 50 μg/mL, about 10 μg/mL to about 100 μg/mL, or about 10 μg/mL to about 50 μg/mL). In some embodiments, nigericin (or an analog thereof) is added to the cell culture medium to reach a concentration of about 50 μg/mL. In some embodiments, nigericin (or an analog thereof) is added to the cell culture medium to reach a concentration of at least about 50 μg/mL. In some embodiments, AG 879 (or an analog thereof) is added to the cell culture medium to reach a concentration of about 50 μg/mL. In some embodiments, AG 879 (or an analog thereof) is added to the cell culture medium to reach a concentration of at least about 50 μg/mL.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### Example

### A. Materials and Methods

### Cell Culture, Chemicals and Antibodies

Human osteosarcoma U2OS cells obtained from the Kroemer lab were maintained at 37° C. in a humidified incubator with 5% CO₂ in Dulbecco's Modified Eagle Medium (DMEM) supplemented with 10% Fetal Bovine Serum (FBS), 1% penicillin-streptomycin and 1% HEPES (Gibco, Carlsbad, CA). CHO-K1 (CCL-61) cells were obtained from the ATCC and cultured at 37° C. in a humidified incubator with 5% CO₂ in Nutrient Mixture F-12 Ham's (Sigma, Saint-Louis, MO) supplemented with 10% FBS and 2 mM L-Glutamine (Gibco). PD-L1-expressing CHO cells were cultured in similar conditions in medium supplemented with 7.5 μg/mL puromycin (Gibco). The Prestwick, the BML-2840 ICCB Known Bioactives and the Autophagy Compounds libraries were purchased from Prestwick Chemicals (Illkirch, France), Enzo Life Sciences (Farmingdale, NY), and TargetMol (Boston, MA), respectively. The anti-streptavidin (Santa Cruz Biotechnology, Dallas, TX), anti-calreticulin (Abcam, Cambridge, UK), anti-SBP (Santa Cruz Biotechnology), anti-human heavy and light IgG chains (Invitrogen) and the anti-GFP (Cell Signaling, Danvers, MA) antibodies were and used at 1:500, 1:200, 1:500, 1:5,000 and 1:1,000 dilutions, respectively, in Western blotting and immunofluorescence experiments. The reference anti-human PD-L1 antibody used in FACS experiments was produced in Sanofi labs from cells transfected with plasmid constructs based on the published Atezolizumab heavy and light chains sequences available on IMGT website (imgt.org) (Lefranc et al., Methods in Molecular Biology (2018) 1827:35-69. The secondary Alexa Fluor 568 and 488-conjugated anti-mouse and rabbit IgG antibodies (Life Technologies, Carlsbad, CA, USA) were used at 1:1,000 and 1:500 dilutions, respectively, in immunofluorescence and FACS experiments. The secondary HRP-conjugated goat anti-mouse and rabbit IgG (Southern Biotech, Birmingham, AL) antibodies were used at a 1:5000 dilution in Western blotting experiments. The anti-human kappa light chain antibody (Thermo Fisher Scientific, Waltham, MA) was used at a 1:4,000 dilution in ELISA.

### Constructs Used for the RUSH Antibody Secretion System

The streptavidin-KDEL plasmid was described in Zhao et al., Sci Rep. (2018) 8:14966. The amino acid sequences of the heavy and light chains of the anti-PD-L1 atezolizumab antibody available on IMGT were used to generate nucleic acid sequences adapted to expression in human cells. Sequences encoding a furin-cleavable site (AVSKERSKRSP) (SEQ ID NO:63), a linker (G₄S) 3 (SEQ ID NO:1) enhanced Green Fluorescent Protein (eGFP) and a streptavidin binding peptide (SBP) were added in this order, to the 3′ end of the anti-PD-L1 heavy chain coding sequence. Anti-PD-L1 heavy and light chains constructs were manufactured by GeneArt (Ratisbonne, Germany) and provided in a shuttle vector. The heavy and light chains sequences were then cloned into the multiple cloning sites 2 and 1, respectively, of the pVITRO1 plasmid (Invivogen, San Diego, CA) using the NEBuilder® assembly kit (New England Biolabs, Ipswich, MA). The integrity of the construct thereafter referred to as anti-PD-L1 IgG RUSH plasmid was confirmed by sequencing (Eurofins, Luxemburg).

### Establishment of the RUSH-Antibody Cell Line

U2OS cells were transfected with the streptavidin-KDEL plasmid using Lipofectamine 2000 (Thermo Fisher Scientific) and selected in medium supplemented with G₄₁₈ (400 μg/mL, Gibco-Invitrogen) for 2 weeks. Limiting dilution in 96-well plates was used for single cell isolation from the selected pool. Clones were amplified and selected based on streptavidin immunofluorescence. Clone #BD6 showing the highest streptavidin expression was further expanded in medium supplemented with G₄₁₈ (200 μg/mL) and transfected with the anti-PD-L1 IgG RUSH plasmid using Lipofectamine 2000 and selected in medium supplemented with G₄₁₈ (200 μg/mL) and hygromycin (100 μg/mL, Santa Cruz Biotechnology) for 2 weeks. Limiting dilution in 96-well plates was used for single cell isolation from clone #BD6-derived selected pool. Clones were amplified and selected based on GFP fluorescence and clone #7 was selected for further characterization and screening campaigns.

### Immunofluorescence

U2OS clone #7 cells were seeded in black 96-well imaging plates (Greiner bio one, Kremsmünster, Austria). After 24 h, the medium was removed, cells were rinsed with 37° C. pre-heated PBS and fixed in PBS supplemented with 4% paraformaldehyde (PFA, Sigma) and 1 μg/mL Hoechst (Life Technologies, Carlsbad, CA, USA) for 20 min under agitation at room temperature (RT). Cells were rinsed with PBS before addition of a 5% FBS/0.3% Triton X-100/PBS permeabilizing solution for 30 min under agitation at RT. Cells were rinsed with PBS and incubated overnight under agitation at 4° C. with the primary antibody in 1% BSA/PBS. Cells were washed with PBS and incubated for 45 min under agitation at RT with the secondary antibody in 1% BSA/PBS. Finally, cells were washed, and processed for image acquisition using an IXM XL BioImager (Molecular Devices, Sunnyvale, CA).

### Antibody Expression and Secretion Characterization by Western Blot

U2OS clone #7 cells were seeded in T-175 flasks and kept for 24 h at 37° C. in a humidified incubator with 5% CO₂. The next day, the supernatant was removed, cells were rinsed with 37° C. pre-heated PBS and new serum-free medium containing no biotin, 40 μM biotin, or 40 μM biotin together with 100 μM of furin inhibitor I (Sigma) was added to the flask for 4 h. At the end of the incubation time, cells were harvested and lysed in RIPA buffer (Thermo Fisher Scientific) supplemented with protease and phosphatase inhibitors (Roche, Basel, Switzerland). Additionally, cell culture supernatants were collected and concentrated ~100-fold in Amicon 50 kDa filter unit (Sigma) according to the manufacturer's instructions. Cell lysate and concentrated supernatant protein concentrations were determined using the BCA™ protein assay kit (Thermo Fisher Scientific). Ten μg of total protein content from lysates and supernatants were resolved on NuPAGE™ 4-12% Bis-Tris gels (Invitrogen, Carlsbad, CA, USA) under reducing conditions and transferred to a nitrocellulose membrane (BioRad) using BioRad system. The membranes were blocked for 1 h with 0.01% Tween-20/5% non-fat dry milk/TBS and the primary antibodies were added overnight under agitation at 4° C. The membranes were rinsed with 0.01% Tween-20/5% non-fat dry milk/TBS and incubated for 1 h with secondary antibodies under agitation at 4° C. The membranes were then washed 3 times for 5 min with 0.01% Tween-20/TBS and peroxidase activity was evaluated with Amersham ECL Primer Western Blotting Detection Reagent (GE Healthcare, Little Chalfont, UK) on ImageQuant™ LAS4000 (GE Healthcare).

### Flow Cytometric Validation of Antibody Reactivity

U2OS clone #7 cell culture supernatants were prepared as described above and used to assess the binding of the RUSH antibody to PD-L1-expressing cells by flow cytometry. Three million CHO-K1 and CHO PD-L1 cells were harvested, rinsed with PBS, and incubated for 30 min at 4° C. under agitation in the dark with either 150 μL of the recombinant atezolizumab prepared in Sanofi research labs or concentrated U2OS clone #7 cell culture supernatant. Cells were then rinsed with PBS and secondarily stained for 30 min at 4° C. under agitation in the dark with an Alexa Fluor 647-coupled anti-human IgG antibody before flow cytometry analysis of 100 μL solution on a MACSQuant® (Miltenyi Biotec, Bergisch Gladbach, Germany).

### High-Throughput Compound Screening for Modulators of Protein Secretion

U2OS clone #7 cells were seeded in black 384-well imaging plates (Greiner bio one), 2,000 cells/well, and incubated for 24 h at 37° C. in a humidified incubator with 5% CO₂. Cells were treated on the next day with the compounds from either the Prestwick library at 20 μM or 10 μM, the ICCB Known Bioactives library at 1/100^{e} or 1/1000^{e}, or the autophagy compound library at 10 μM or 1 μM for 4 h. At the end of the incubation time, biotin was added at 40 μM for 1 h (untreated cells and DMSO-treated cells were used as control). Cells were then fixed as described above and processed for subsequent automated image acquisition using a robot-assisted IXM XL BioImager (Molecular Devices, Sunnyvale, CA, USA) equipped with a Sola light source (Lumencor, Beaverton, OR, USA), adapted excitation and emission filters (Semrock) a 16-bit monochrome sCMOS PCO.edge 5.5 camera (PCO, Kelheim, Germany). A 20× PlanAPO objective (Nikon, Tokyo, Japan) was used to acquire a minimum of 4 view fields in each well. The acquired images were processed using the open-access R software (https://www.r-project.org) with the help of freely available packages EBImage (available on the Bioconductor repository https://www.bioconductor.org), and RBioFormats (https://github.com/aoles/RBioFormats), and the custom packages MetaxpR (https://github.com/asauvat/MetaxpR) and MorphR (https://github.com/kroemerlab/MorphR). Nuclei were first detected using Hoechst 33342 signal and used as a marker to detect cytoplasmic areas based on GFP signal, allowing for the evaluation of cytoplasmic GFP intensity. Data were thereafter extracted and statistically evaluated using R (https://www.r-project.org). Data were normalized using negative and positive controls, reduced and transformed into a Z-score. The experiments were repeated manually at low scale with selected drugs to confirm their effects on secretion, in this second set of assays, U2OS clone #7 cells incubated with secretion inhibitors were fixed and stained with Hoechst as described above for imaging and viability determination based on cell counts.

### ELISA

U2OS clone #7 cells were seeded in 6-well plates and kept for 24 h at 37° C. in a humidified incubator with 5% CO₂. The next day, the supernatants were removed, cells were rinsed with 37° C. pre-heated PBS and new medium containing the chemical treatment was added for 4 h. At the end of the incubation time, no biotin or 40 μM biotin was added to the flask for 30 min (inhibitory selected molecules) or 1 h (activator selected molecules). Then supernatants were collected and used for the ELISA assay as follows. MaxiSorp 96-well plates (Thermo Fisher Scientific) were coated with 0.5 μg/mL hFc-human-PD-L1 (R&D Systems, Minneapolis, MN) in PBS for 5.5 h at RT. Coating solution was then removed and 2% BSA/PBS blocking solution was added overnight at 4° C. The next day, wells were washed 3 times with 0.5% BSA/0.05% Tween-20/PBS solution. Supernatants were added after a ⅓ dilution in 0.5% BSA/0.05% Tween-20/PBS, for 2.3 h at RT. Wells were washed 3 times with 0.5% BSA/0.05% Tween-20/PBS solution. Anti-human kappa light chain HRP-coupled antibody was added diluted in 0.5% BSA/0.05% Tween-20/PBS at 1/4000^{e} for 2.3 h at RT. Wells were washed 3 times with 0.5% BSA/0.05% Tween-20/PBS solution before incubation with substrate reagents pack (R&D Systems) for 15 min in the dark. Fifty μL of STOP solution (R&D Systems) were then added to each well and the absorbance in each well was subsequently measured at 450 nm, and 635 nm for background subtraction, using a VICTOR™ X4 (PerkinElmer, Waltham, MA).

### Statistics

Unless otherwise specified, data are reported as mean±SD of two replicates in a minimum of three independent experiments. Statistical significance was assessed using Mann-Whitney U-test.

### B. Results

### Design of an Antibody RUSH System

The RUSH system is composed of a hook which is streptavidin, and a bait which is a protein fused to a streptavidin-binding peptide (SBP). When these two molecules are co-expressed, the bait is sequestered by the hook due to the high-affinity streptavidin/SBP interaction (K_{D} in the range of 10⁻⁸ to 10⁻⁹ M) (Barrette-Ng et al., Acta Crystallographica. Section D, Biological Crystallography (2013) 69:879-87), and released upon addition of biotin, which outcompetes SBP due to its higher affinity for streptavidin (K_{D} in the range 10⁻¹⁴ M) (Delgadillo et al., PLOS One (2019) 14:e0204194). Here we have used this cell-based assay to monitor the secretion kinetics of an antibody (the bait) and screen small compound libraries to identify secretion modulators. Because antibodies are secreted through the conventional pathway, the streptavidin hook was fused to a KDEL peptide to ensure its retention in the endoplasmic reticulum (ER) through the interaction with KDEL-receptors present in the ER (Capitani, FEBS letters (2009) 583:3863-71).

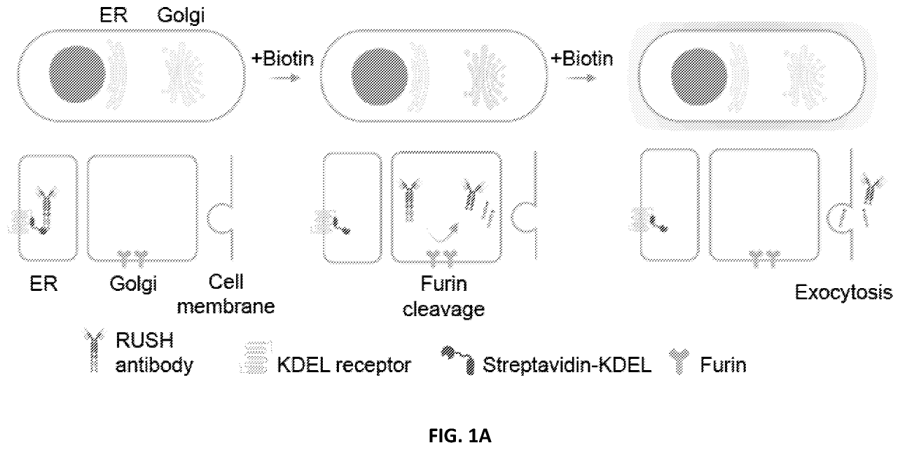

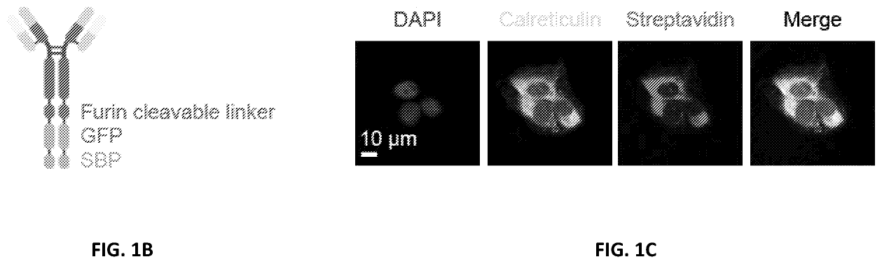

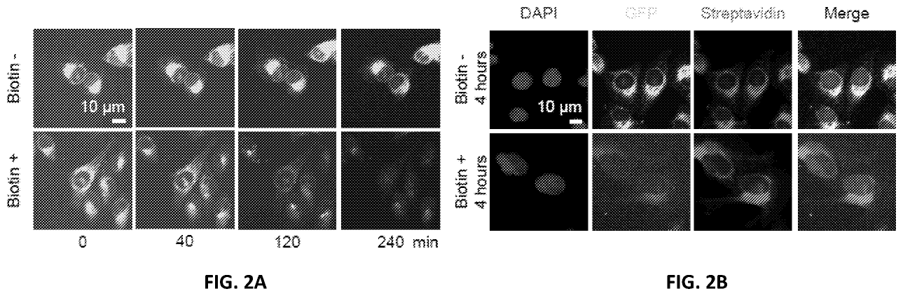

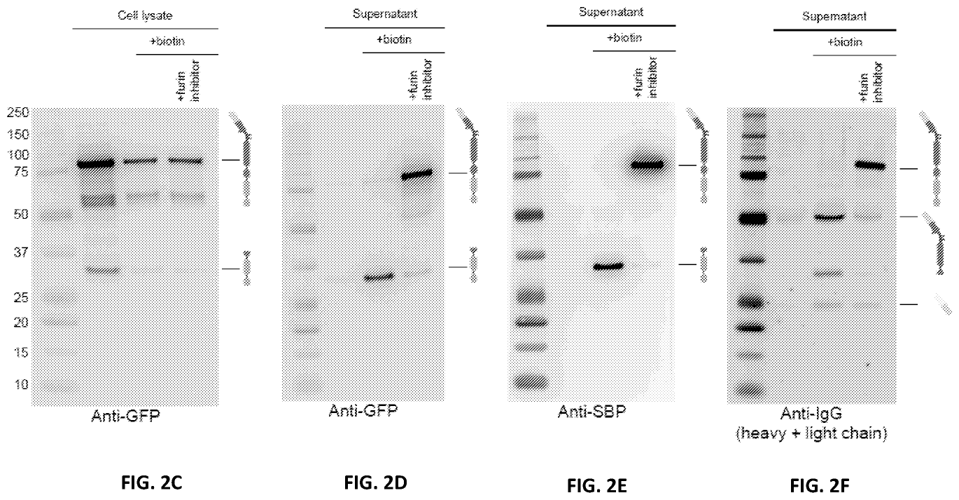

The antibody was also modified to fit the purpose of the assay. A polycistronic vector was used, that encodes both the heavy and light (kappa) chains of a human PD-L1-specific antibody (Zhang et al., Oncotarget (2017) 8:90215-24). The C-terminus of the heavy chain was fused to a (Gly₄Ser)₃ linker, a furin-cleavable site, the green fluorescent protein (GFP) and SBP (FIG. 1B). We reasoned that the fusion molecule (thereafter called RUSH antibody) would assemble into mature homodimers upon protein synthesis and remain in the ER due to its interaction with the streptavidin KDEL hook until addition of biotin, which would trigger the RUSH antibody's release and maturation in the Golgi en route to the extracellular space (FIG. 1A). The hook and bait expressing clone was obtained by U2OS cell transfection with the hook-encoding construct, and the selection of a clone with high hook expression levels in order to ensure efficient retention of the bait and minimize leakiness in the absence of biotin. As shown in FIG. 1C, Clone #BD6 staining with streptavidin-specific antibodies reveals the colocalization of the hook with the prototypic ER-sessile protein calreticulin (Nomura et al., Histochemistry and Cell Biology (2011) 135:531-8). Clone #BD6 was further transfected with the RUSH antibody-encoding vector followed by single cell cloning and the selection of the hook/bait clone used in this study (clone #7). This clone was selected based on two criteria: (i) a bright GFP-dependent fluorescent signal indicating high abundance of the RUSH antibody and (ii) discrete localization of the GFP signal in the perinuclear area indicating ER retention (FIG. 2A). Fluorescence video-microscopic observation of cells from clone #7 revealed that GFP remains in the same subcellular compartment over the 2 hours of acquisition.

On the contrary, the addition of biotin resulted in the relocation of the GFP signal towards discrete puncta that correspond to the Golgi apparatus within 40 to 120 min, and a complete cytoplasmic GFP signal decay within 4 h most likely due to the release of the fusion protein into the extracellular space (FIG. 2A). The GFP signal co-localized with streptavidin at baseline and addition of biotin did not modify this pattern, confirming the retention of the hook in the ER regardless of the presence or absence of biotin in the medium. On the contrary the GFP/streptavidin co-localization was lost 4 h after addition of biotin (FIG. 2B).

These results confirm the expression of the bait, its retention in the ER through the hook and suggest its progressive release in the extracellular space upon addition of biotin.

### Biochemical Validation of the Antibody RUSH System

In the next step we proceeded to the biochemical validation of clone #7 before its use in a screening campaign. To this aim, we characterized the molecular species synthesized and released by clone #7 cultured with or without biotin and a furin protease inhibitor. As can be seen in FIG. 2C, GFP-specific antibodies used in western blotting experiments revealed the presence of a major molecular species of ~80 kDa and two minor species of ~55 and ~35 kDa in the lysate of cells grown in the absence of biotin. The 80 kDa mass is compatible with the full-length heavy chain of the RUSH antibody (theoretical mass of 84 kDa) while the 35 kDa mass is compatible with the GFP-SBP C-terminal fragment of the heavy chain which is released upon cleavage by furin (theoretical mass of 32 kDa). Similar analysis performed on the cell culture supernatant with GFP, SBP and human IgG-specific antibodies only revealed trace amounts of recombinant protein in the absence of biotin (FIG. 2D to 2F). This is consistent with the minor processing observed in cell lysates and therefore efficient retention of the RUSH antibody in pre-Golgi intracellular compartments as documented by immunofluorescence (FIGS. 2A and B).

When cells were cultured in the presence of biotin, the Western blotting pattern in the cell lysate was unchanged except that the intensity of the signal was lower, which was concomitant with the detection of recombinant species in the cell cultures supernatant (FIG. 2C to 2F). This demonstrates that the intracytoplasmic GFP decay observed by immunofluorescence in FIGS. 2A and 2B is a direct consequence of protein release in the extracellular space. Moreover, the molecular masses of the material detected in the supernatant suggest an almost full processing of the released RUSH antibody. This is further confirmed by the comparable patterns obtained in cell lysates and in the cell culture supernatant when cells are grown in the presence of furin inhibitor. It can therefore be concluded that the RUSH antibody is released through the Golgi network and processed upon addition of biotin.

All together, these findings demonstrate that clone #7 allows for the biotin-stimulated release, as well as the furin-dependent proteolytic maturation, of the RUSH antibody.

### Immunological Validation of the Antibody RUSH System

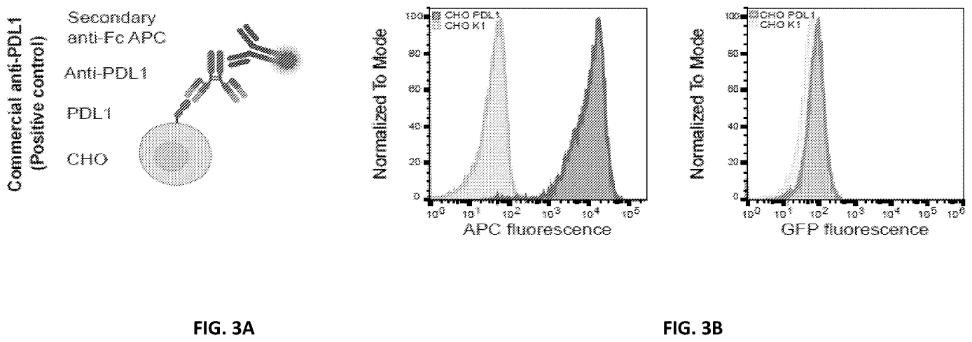

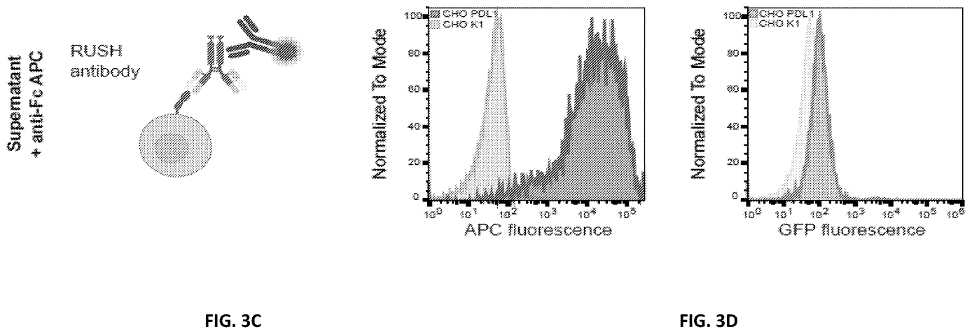

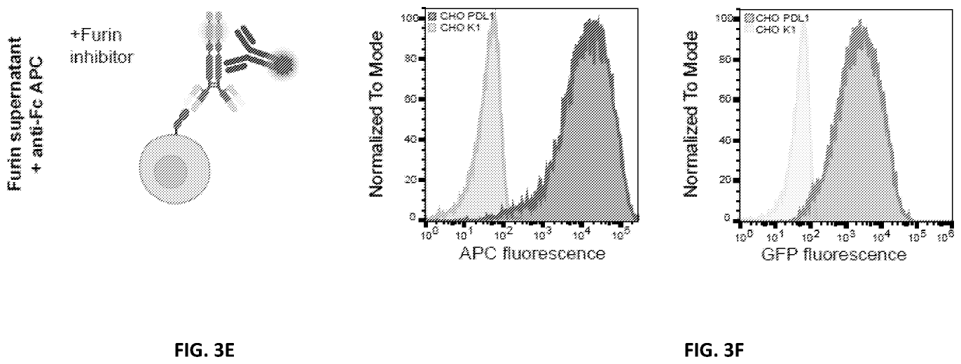

Besides the mechanistic validation of clone #7 in terms of antibody production, retention, maturation and release upon addition of biotin, we also addressed the PD-L1 binding properties of the secreted RUSH antibody. An ELISA demonstrated the specificity of the RUSH antibody to immobilized recombinant PD-L1 (not shown). We further investigated the RUSH antibody binding properties to PD-L1 in its native conformation when it is presented at the plasma membrane. To this aim, clone #7 was incubated with biotin and the cell culture supernatant was assayed for binding on PD-L1^{pos} and PD-L1^{neg} CHO cells by FACS. As can be seen in FIGS. 3A and 3B, native, recombinant anti-PD-L1 binding to PD-L1^{pos} (CHO-PDL1) but not PD-L1^{neg} CHO (CHO-K1) cells can be documented, which validates the use of such system for further characterization of the RUSH antibody. Similarly, the supernatant of clone #7 cultured in the presence of biotin demonstrated binding activity to CHO-PDL1 cells but not to CHO-K1 cells (FIGS. 3C and D), indicating that the released RUSH antibody conserved its antigen-binding properties despite the presence of extra amino acids at its C-terminus, left after processing by furins. Of note, PD-L1 binding was also detectable in the supernatant of clone #7 cultured in the presence of biotin and a furin protease inhibitor (FIGS. 3E and 3F). In these conditions, binding is detected through both the secondary staining antibody and the GFP moiety of the unprocessed RUSH antibody which was not detectable with the native antibody (FIG. 3B) and only very slightly detected in the case of the supernatant of clone #7 cultured with biotin only (FIG. 3D).

Altogether, these observations indicate that the clone #7 is fit for both screening campaigns and further antibody characterization in PD-L1-binding assays.

### Pharmacological Screen for Modulators of Biotin-Induced Antibody Release

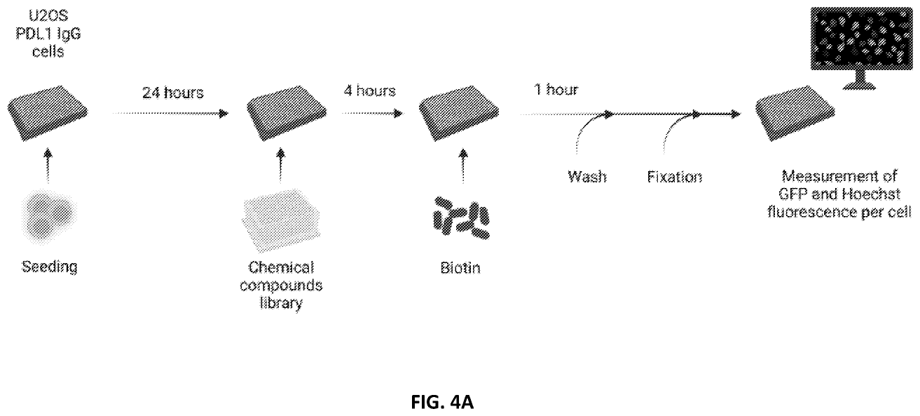

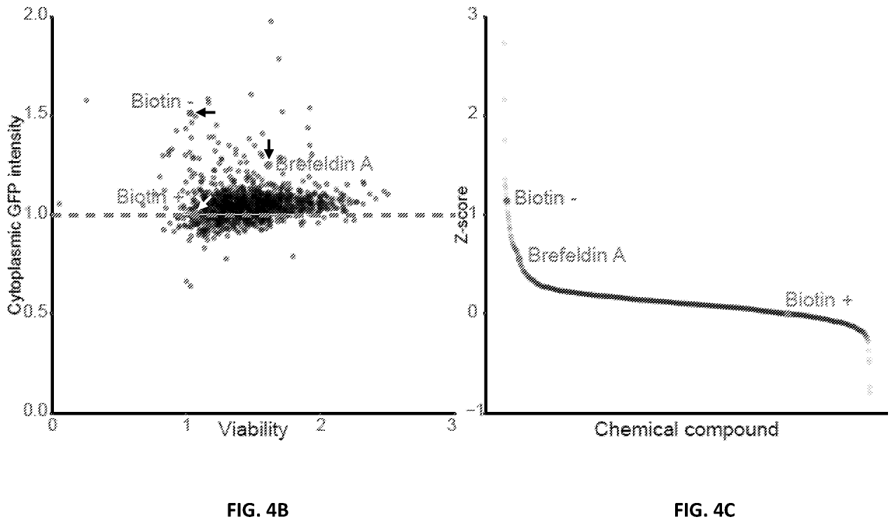

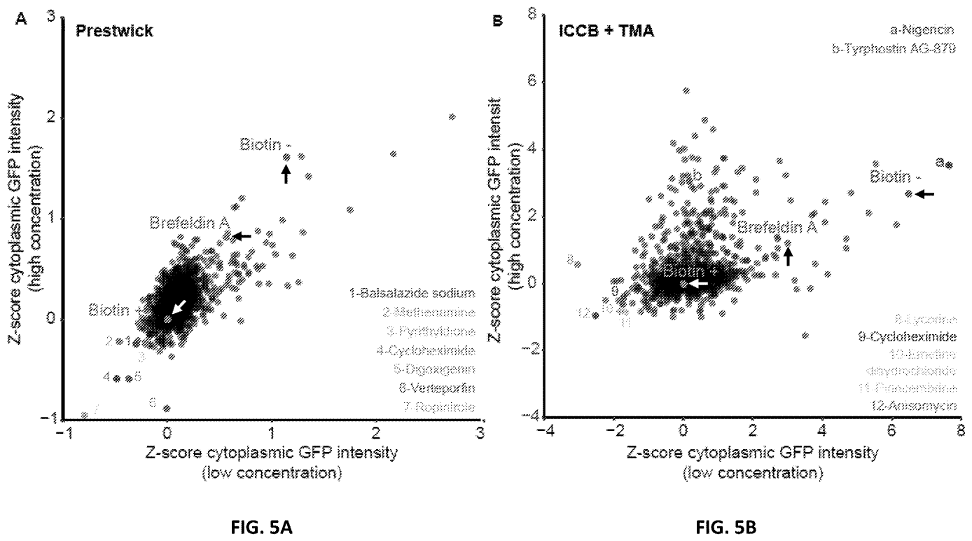

We next sought to use clone #7 in screening campaigns for the identification of drugs capable of enhancing or inhibiting antibody secretion. For this, clone #7 was cultured in 384 well plates and treated for 4 h with drugs from chemical libraries, followed by the addition of biotin for 1 h and the determination of intra-cytoplasmic GFP signal analysis (FIG. 4A). Cell counting was facilitated by nuclear counterstaining with Hoechst 33324 in order to assess viability and distinguish drug effects on the secretion kinetics from direct cytotoxicity and select relevant drugs for further characterization. Two independent screening campaigns were performed to test a total of 2,300 drugs at 2 different concentrations (i.e., a low concentration and a 10-fold higher concentration referred to as “low” and “high” dose as described in the Materials & Methods section).

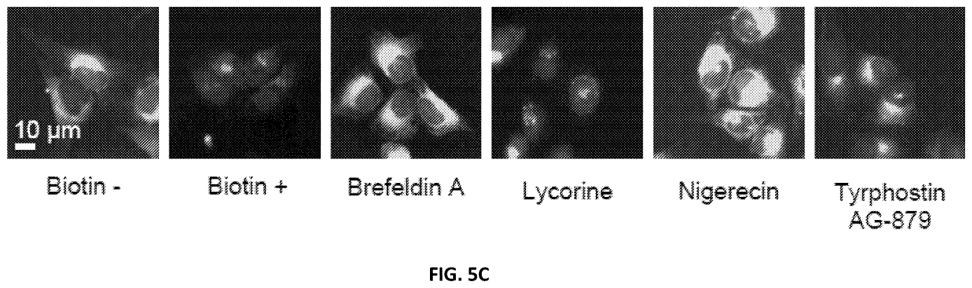

The biotin-induced decrease of the cellular GFP signal was influenced by a minority of compounds from various chemical libraries, as indicated by plotting of primary data (FIG. 4B) or Z-score analyses (FIG. 4C). As an internal control, brefeldin A which is an inhibitor of conventional Golgi-dependent protein secretion (Misumi et al., J Biol Chem. (1986) 261:11398-403) inhibited the biotin-induced decrease of the GFP signal (FIGS. 4B and C). Plotting of the Z-scores obtained at the low and high concentrations for each compound (FIGS. 5A and B) led to the identification of several compounds that consistently decreased the fluorescent signal, therefore acting as secretion activators. In order to select candidates to be further characterized, the 20 best activators identified at either the low or high concentration were listed and among these, activators working at both concentrations were shortlisted.

In the Prestwick library, balsalazide sodium (not commercially available anymore), methenamine, pyrithyldione, cycloheximide, digoxigenin, verteporfin and ropinirole fulfilled these criteria (FIG. 5A). Verteporfin was eliminated due to its phototoxicity that leads to GFP bleaching (data not shown). In a combined screen involving compounds contained in two additional libraries (ICCB Known Bioactives and TargetMol Autophagy), several additional compounds, anisomycin, emetine and pinocembrine, were active at both low and high concentrations. Additionally, although lycorine was not among the 20 most active compounds at the high concentration, it was more efficient than any other compound at low concentration and was therefore selected (FIG. 5B).

Several drugs were also identified that inhibited the biotin-induced cytoplasmic GFP signal decay, therefore acting as potential secretion inhibitors, among which nigericin and tyrphostin AG-879 were selected for further characterization (FIG. 5B) because they have not been identified previously (Zhao et al., Sci Rep. (2018) 8:14966).

### Validation of Stimulators and Inhibitors of the Biotin-Induced Antibody Release

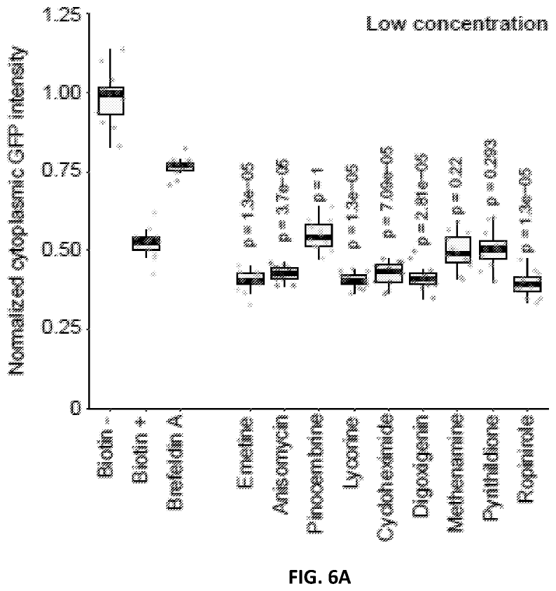

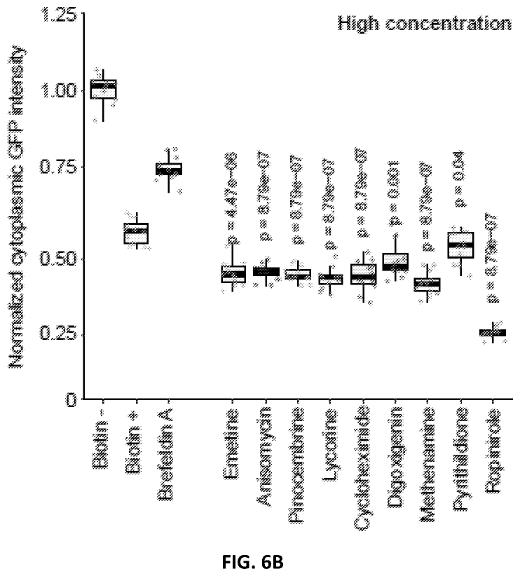

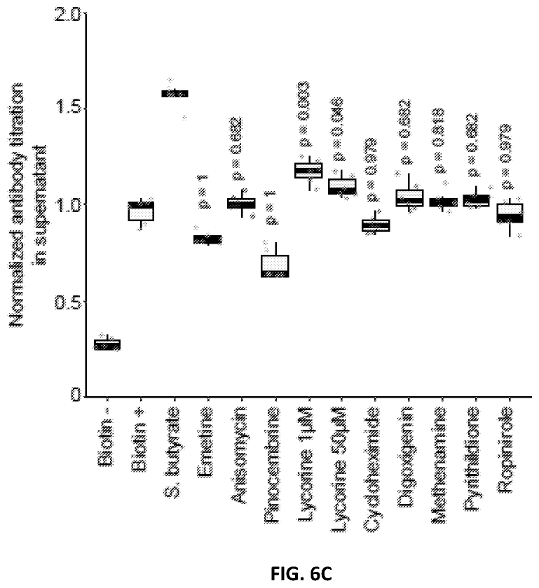

Medium- and high-throughput screenings often yield false positive and negative hits, calling for independent validation in low-throughput experiments with orthogonal technologies. In a first round of experiments, we evaluated the effects of shortlisted compounds with potential antibody secretion enhancer activity. Compounds identified in the screening campaigns were tested again on clone #7 at a low concentration (FIG. 6A) or a ten-fold higher concentration (FIG. 6B) for their ability to increase antibody secretion. This experiment shows that all selected hits were active at the highest concentration, which confirms the results of the screening (FIG. 6B). We further characterized these hits in an PD-L1 binding ELISA. In this system, sodium butyrate induced a two-fold increase of RUSH antibody release upon biotin addition, as expected from its effect in protein expression (Jiang and Sharfstein, Biotechnol Bioeng. (2008) 100:189-94; Mimura et al., J Imm Methods (2001) 247:205-16). Among the drugs tested, only lycorine (tested at both 1 and 50 μM) was considered to be active, although its effect was milder than that of sodium butyrate, while all the other compounds failed to display any antibody secretion-stimulatory activity (FIG. 6B).

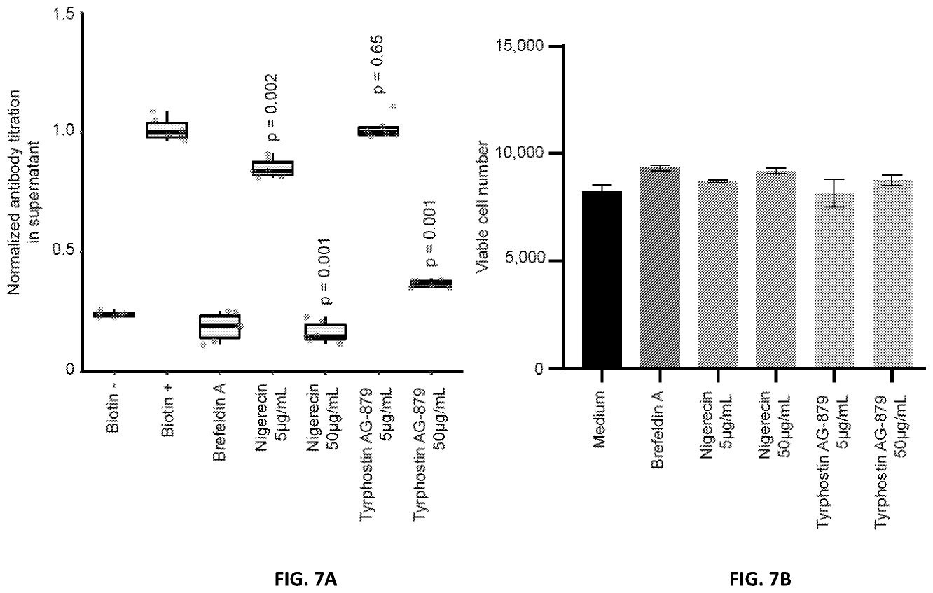

Similarly, potential secretion inhibitors identified during the screening campaigns were also tested in an ad hoc assay. Both nigericin and tyrphostin AG-879 (used at 50 μg/mL) turned out to be as potent as brefeldin A in suppressing antibody secretion (FIG. 7A) without affecting cellular viability (FIG. 7B). Cell imaging further confirms the secretion activator effect of lycorine and the inhibitory effect of nigericin and tyrphostin AG-879 (FIG. 5C).

### C. Summary

In the present study, we provide evidence that, using an antibody-compatible RUSH system, small chemical compounds libraries were successfully screened using a high-throughput fluorescence imaging system for the identification of modulators of conventional antibody secretion. Here we report one novel activator and two novel inhibitors of antibody secretion validated by ELISA antibody titration.

Setting up a RUSH system requires the addition of SBP to the antibody for reversible ER targeting and a fluorescent protein (e.g. GFP) for automatized video microscopy monitoring of antibody secretion. This represents a challenge for antibodies since immunoglobulins and GFP do not normally originate from the same cellular compartment and the addition of foreign sequences to antibodies has been shown to interfere with their secretion and functionality (Haas et al., Methods Mol Biol. (2012) 901:265-76; Luria et al., MAbs. (2012) 4:373-84). The mechanism of IgG heavy and light chain assembly prior to mature antibody secretion posed another challenge. Indeed, while antibody heavy chains require assembly with light chains for secretion, free light chain might be secreted. We therefore opted to retain the RUSH antibody through its heavy chain in order to confidently monitor the secretion of fully assembled antibodies. All constraints considered, the RUSH machinery was added to the C-terminus of the anti-PD-L1 heavy chain. A furin cleavage site was also added between the heavy chain and the RUSH construct for the removal of the SBP and GFP sequences past the Golgi network, that might affect the functions of the antibody otherwise. Our data show that the uncleaved full length antibody retained its specificity and was detectable through its GFP moiety.

Among the two unique compounds that caused the retention of the RUSH antibody in the cytoplasm, tyrphostin AG-879 is characterized as a tyrosine kinase inhibitor (IC50=10 μM) and could be validated as an inhibitor of antibody secretion when it was used at a concentration of 50 μg/mL. Nigericin has also been confirmed as a secretion inhibitor when used at a concentration of 50 μg/mL.

Our screening campaigns also led to the identification of candidate molecules that increased antibody secretion in the biotin-stimulated RUSH system because they decreased the intracytoplasmic GFP fluorescence signal, among which 8 compounds (anisomycin, cycloheximide, digoxigenin, lycorine, methenamine, ropinirole, pinocembrine and pyrithyldione) were selected for further characterization by ELISA. This assay measures the RUSH antibody binding to its immobilized PD-L1 target and was used to measure antibody titers in cells treated with candidate molecules. Unexpectedly, among these 8 compounds that consistently decreased intracytoplasmic GFP fluorescence, which was interpreted as accelerated antibody release, only lycorine was shown to increase RUSH antibody titers in treated cell culture supernatant. On the contrary, the fact that lycorine was validated in both assays and in several independent experiments strongly suggests its stimulatory effect on antibody secretion in our system.

Lycorine likely has multiple effects on cell biology with several cellular targets and a proposed utility for rather diverse indications including cancer (Roy et al., Biomedicine & Pharmacotherapy=Biomedecine & Pharmacotherapie (2018) 107:615-24), fungal infection (Zhao et al., Biotechnology Letters (2021) 43:1503-12) and Alzheimer's disease (Kola et al., Int J Mol Sci. (2023) 24 (3): 2500); however, no effect on secretion is published to the best of our knowledge.

1. A method of inhibiting protein secretion from a eukaryotic cell, comprising contacting the cell with nigericin or tyrphostin AG-879, or an analog thereof.

2. The method of claim 1, wherein the cell is in a cell culture.

3. The method of claim 2, wherein the contacting comprises adding nigericin, tyrphostin AG-879, or analog to the cell culture.

4. The method of claim 3, wherein the nigericin, tyrphostin AG0879, or analog is added to the cell culture to reach a concentration of about 1 μg/mL to about 100 μg/mL, optionally about 5 μg/mL to about 50 g/mL.

5. The method of claim 3, wherein the nigericin, tyrphostin AG0879, or analog is added to the cell culture to reach a concentration of at least about 50 μg/mL; optionally, wherein the nigericin, tyrphostin AG0879, or analog is added to the cell culture to reach a concentration of about 50 μg/mL.

6. A method of increasing protein secretion from a eukaryotic cell, comprising contacting the cell with lycorine or an analog thereof.

7. The method of claim 5, wherein the eukaryotic cell is a recombinant cell engineered to express a recombinant protein.

8. The method of claim 6, wherein the recombinant protein is an enzyme for enzyme replacement therapy, or an antibody.

9. The method of any one of claims 5-7, wherein the cell is in a cell culture.

10. The method of claim 8, wherein the contacting comprises adding lycorine or analog to the cell culture.

11. The method of claim 9, wherein the lycorine or analog is added to the cell culture to reach a concentration of about 0.1 μM to about 100 μM, optionally about 1 μM to about 50 μM.

12. The method of claim 9, wherein the lycorine or analog is added to the cell culture to reach a concentration of at least about 1 μM.

13. The method of claim 9, wherein the lycorine or analog is added to the cell culture to reach a concentration of about 1 μM or of about 50 μM.

14. The method of any one of the preceding claims, wherein the eukaryotic cell is a mammalian cell, an insect cell, or a yeast cell.

15. The method of claim 11, wherein the cell is a mammalian cell, optionally a CHO cell, a NSO cell, a BHK cell, a 293 cell, a HeLa cell, a HEK cell, or a Sp2/0 cell.


SEQUENCE LISTING (63 sequences)

SEQ ID NO: 1 - protein, 5 aa, synthetic construct
  REGION 1..5: note = The entire sequence of amino acids 1-5 can be repeated 1 to 10 times
GGGGS

SEQ ID NO: 2 - protein, 183 aa, synthetic construct
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 3 - protein, 4 aa, synthetic construct
KDEL

SEQ ID NO: 4 - protein, 4 aa, synthetic construct
KTEL

SEQ ID NO: 5 - protein, 4 aa, synthetic construct
HNEL

SEQ ID NO: 6 - protein, 4 aa, synthetic construct
KEDL

SEQ ID NO: 7 - protein, 4 aa, synthetic construct
HDEL

SEQ ID NO: 8 - protein, 4 aa, synthetic construct
KEEL

SEQ ID NO: 9 - protein, 4 aa, synthetic construct
QEDL

SEQ ID NO: 10-12 - skipped (3 entries)

SEQ ID NO: 13 - protein, 7 aa, synthetic construct
  VARIANT 6: note = X can be any amino acid
EFFDAXE

SEQ ID NO: 14 - protein, 46 aa, synthetic construct
MAGARRGWRLAPVRRGVCGPRARPLMRPLWLLFAVSFFGWTGALDG

SEQ ID NO: 15 - protein, 15 aa, synthetic construct
MGSLWSKISQLFVDA

SEQ ID NO: 16 - protein, 28 aa, synthetic construct
MRVKEKYQHLWRWGWRWGTMLLGMLMIC

SEQ ID NO: 17 - protein, 21 aa, synthetic construct
MRLSWFRVLTVLSICLSAVAT

SEQ ID NO: 18 - protein, 29 aa, synthetic construct
MDPVVVLGLCLSCLLLLSLWKQSYGGGKL

SEQ ID NO: 19 - protein, 35 aa, synthetic construct
HALSYWKPFLVNMCVATVLTAGAYLCYRFLFNSNT

SEQ ID NO: 20 - protein, 44 aa, synthetic construct
SPRGSRPSWGPTDPRRRSRNLGKVIDTLTCGFADLMGYIPLVGA

SEQ ID NO: 21 - protein, 24 aa, synthetic construct
MALWMRFLPLLALLVLWEPKPAQA

SEQ ID NO: 22 - protein, 17 aa, synthetic construct
MEAMWLLCVALAVLAWG

SEQ ID NO: 23 - protein, 27 aa, synthetic construct
IPHDLCHNGEKSKKPSKIKSLFKKKSK

SEQ ID NO: 24 - protein, 69 aa, synthetic construct
RPSNNNTTTTTTTDATQPHHHHHHHRHRDAGVKNVTNNSKTTESSSSSSAAKEKPKHKKGLLHKLKKKL

SEQ ID NO: 25 - protein, 40 aa, synthetic construct
VVQAITFIFKSLGLKCVQFLPQVMPTFLNVIRVCDGAIRE

SEQ ID NO: 26 - protein, 21 aa, synthetic construct
TFCSTALLITALALVCTLLYL

SEQ ID NO: 27 - protein, 18 aa, synthetic construct
GVMLGSIFCALITMLGHI

SEQ ID NO: 28 - protein, 23 aa, synthetic construct
FLAAFVVMLCITIVTIVVYIYLV

SEQ ID NO: 29 - protein, 29 aa, synthetic construct
WGVLAGIAYFSMVGNWAKVLVVLLLFAGV

SEQ ID NO: 30 - protein, 10 aa, synthetic construct
TAPASINTPN

SEQ ID NO: 31 - protein, 22 aa, synthetic construct
WWQLTLGAICALPLAGLLACCA

SEQ ID NO: 32 - protein, 37 aa, synthetic construct
DEKTTGWRGGHVVEGLAGELEQLRARLEHHPQGQREP

SEQ ID NO: 33 - protein, 9 aa, synthetic construct
AWRHPQFGG

SEQ ID NO: 34 - protein, 18 aa, synthetic construct
AWRHPQFGGAWRHPQFGG

SEQ ID NO: 35 - protein, 27 aa, synthetic construct
AWRHPQFGGAWRHPQFGGAWRHPQFGG

SEQ ID NO: 36 - protein, 8 aa, synthetic construct
WSHPQFEK

SEQ ID NO: 37 - protein, 16 aa, synthetic construct
WSHPQFEKWSHPQFEK

SEQ ID NO: 38 - protein, 24 aa, synthetic construct
WSHPQFEKWSHPQFEKWSHPQFEK

SEQ ID NO: 39 - protein, 8 aa, synthetic construct
WSGPQFEK

SEQ ID NO: 40 - protein, 16 aa, synthetic construct
WSGPQFEKWSGPQFEK

SEQ ID NO: 41 - protein, 24 aa, synthetic construct
WSGPQFEKWSGPQFEKWSGPQFEK

SEQ ID NO: 42 - protein, 10 aa, synthetic construct
WHRWPWLVSG

SEQ ID NO: 43 - protein, 20 aa, synthetic construct
WHRWPWLVSGWHRWPWLVSG

SEQ ID NO: 44 - protein, 30 aa, synthetic construct
WHRWPWLVSGWHRWPWLVSGWHRWPWLVSG

SEQ ID NO: 45 - protein, 10 aa, synthetic construct
WHWWYWALDR

SEQ ID NO: 46 - protein, 20 aa, synthetic construct
WHWWYWALDRWHWWYWALDR

SEQ ID NO: 47 - protein, 30 aa, synthetic construct
WHWWYWALDRWHWWYWALDRWHWWYWALDR

SEQ ID NO: 48 - protein, 8 aa, synthetic construct
  VARIANT 2: note = X can be any amino acid
  VARIANT 7: note = X can be G or E
  VARIANT 8: note = X can be G, R or K
WXHPQFXX

SEQ ID NO: 49 - protein, 4 aa, synthetic construct
VEAW

SEQ ID NO: 50 - protein, 4 aa, synthetic construct
DVEA

SEQ ID NO: 51 - protein, 5 aa, synthetic construct
DVEAW

SEQ ID NO: 52 - protein, 5 aa, synthetic construct
  VARIANT 3: note = X can be E, D or G
DVXAW

SEQ ID NO: 53 - protein, 6 aa, synthetic construct
  VARIANT 3: note = X can be E, D or G
DVXAWL

SEQ ID NO: 54 - protein, 6 aa, synthetic construct
DVEAWL

SEQ ID NO: 55 - protein, 7 aa, synthetic construct
  VARIANT 3: note = X can be E, D or G
  VARIANT 7: note = X can be G or D
DVXAWLX

SEQ ID NO: 56 - protein, 8 aa, synthetic construct
  VARIANT 3: note = X can be G or D
  VARIANT 7: note = X can be G or D
  VARIANT 8: note = X can be A or E
DVXAWLXX

SEQ ID NO: 57 - protein, 9 aa, synthetic construct
  VARIANT 3: note = X can be E, D or G
  VARIANT 7: note = X can be G or D
  VARIANT 8: note = X can be A or E
DVXAWLXXR

SEQ ID NO: 58 - protein, 9 aa, synthetic construct
DVEAWLGAR

SEQ ID NO: 59 - protein, 15 aa, synthetic construct
  VARIANT 3: note = X can be E, D or G
  VARIANT 7: note = X can be G or D
  VARIANT 8: note = X can be A or E
DVXAWLXXRVPLVET

SEQ ID NO: 60 - protein, 6 aa, synthetic construct
DRATPY

SEQ ID NO: 61 - protein, 8 aa, synthetic construct
SEKLSNTK

SEQ ID NO: 62 - skipped

SEQ ID NO: 63 - protein, 11 aa, synthetic construct
AVSKERSKRSP